(19) United States
(12) Patent Application Publication
(10) Pub. No.: US 2003/0003061 A1
(43) Pub. Date: Jan. 2, 2003

(54) Stable dentifrice compositions comprising polyphosphate, fluoride, and stannous

(75) Inventors: Jiang Yue, West Chester, OH (US); Sekhar Mitra, Guangzhou (CN); Yujun Li, Kobe (JP); Hsiang-Kuen Mao, Kobe (JP); Baoan Li, Guangzhou (CN)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/218,777

(22) Filed: Aug. 14, 2002

Related U.S. Application Data
(63) Continuation of application No. PCT/US01/07695, filed on Mar. 12, 2001.
(60) Provisional application No. 60/189,178, filed on Mar. 14, 2000.

(51) Int. Cl. A61K 7/18; A61K 7/16

(52) U.S. Cl. 424/52; 424/57

(57) ABSTRACT

Disclosed are oral compositions comprising: (a) an effective amount of one or more linear polyphosphates having an average chain length of about 4 or more; (b) from about 0.15% to about 5% of a fluoride ion source; (c) from about 0.1% to about 15% of a stannous ion source; (d) an effective amount of a buffering agent; (e) from about 6% to about 70% of an abrasive polishing material containing less than 23% calcium; and (f) from about 40% to about 99% of one or more aqueous carriers; wherein the oral composition has a total water content of from about 1% to about 20%.

### CROSS REFERENCE RELATED TO APPLICATIONS

This is a continuation of International Application PCT/US01/07695 with an international filing date of Mar. 12, 2001, which claims benefit of Provisional Application Serial No. 60/189,178 filed Mar. 14, 2000.

### SUMMARY OF THE INVENTION

The present invention relates to a stable oral composition comprising polyphosphate, a fluoride ion source, and a stannous ion source. More specifically, the present invention relates to a single-phase such dentifrice composition.

### BACKGROUND OF THE INVENTION

Dental calculus, or tartar as it is sometimes called, is a deposit which forms on the surfaces of the teeth at the gingival margin. Supragingival calculus appears principally in the areas near the orifices of the salivary ducts; e.g., on the lingual surfaces of the lower anterior teeth and on the buccal surfaces of the upper first and second molars, and on the distal surfaces of the posterior molars.

Mature calculus consists of an inorganic portion which is largely calcium phosphate arranged in a hydroxyapatite crystal lattice structure similar to bone, enamel, and dentine. An organic portion is also present and consists of desquamated epithelial cells, leukocytes, salivary sediment, food debris, and various types of microorganisms.

As the mature calculus develops, it becomes visibly white or yellowish in color unless stained or discolored by some extraneous agent. This is undesirable from an aesthetic standpoint.

Mechanical removal of calculus periodically by the dentist is routine dental office procedure. A variety of chemical and biological agents have also been suggested to retard calculus formation or to remove calculus after it is formed. Pyrophosphate salts are chemical agents known to have the ability to retard calculus formation as described, for example, in U.S. Pat. No. 4,999,184, to Parran, Jr. et al., issued Mar. 12, 1991.

In addition to the pyrophosphate salts, other polyphosphates are also known to help retard calculus formation. U.S. Pat. No. 4,627,977, issued Dec. 9, 1986, to Gaffar et al. discloses the use of linear molecularly dehydrated polyphosphate salts combined with a fluoride ion-providing source and a synthetic linear polymeric polycarboxylate which inhibit enzymatic hydrolysis of the polyphosphate salt in saliva. U.S. Pat. No. 4,247,526, to Jarvis et al., issued Jan. 27, 1981, discloses the use of a pharmaceutically acceptable condensed phosphate salt in addition to dicalcium phosphate dihydrate and trimagnesium phosphate. Although polyphosphate containing oral care products are known, there is a continuing need to develop stable products containing polyphosphates.

Certain polyphosphates, in particular, linear polyphosphates with average chain lengths greater than 4, will significantly react with most ionic fluoride sources in oral compositions and alter the pH of the oral compositions. This reaction compromises the ability of the oral composition to provide stable ionic fluoride and polyphosphate to the oral surfaces. It has been described that sodium monofluorophosphate is a suitable fluoride ion source to be present with polyphosphate in a single-phased dentifrice composition, see WO 98/22080, published on May 28, 1998. However, it remains desirable to have a wider range of soluble fluoride ion source without compromising stability. Fluoride ion is, of course, a well-known caries protection agent.

In addition, these polyphosphates are hydrolytically unstable, as well as highly hygroscopic, i.e., they decompose very rapidly in the presence of water. Thus, the use of expensive dual compartment dispensers to keep the polyphosphate separated from the aqueous components of the formulation until just prior to use is generally necessary, to ensure product stability. This generally increases the cost of the formulation to consumers; which to some consumers, especially those in developing countries and/or those with less access to professional dental office prophylaxis, may make such products less desirable to consumers.

Stannous (II) ion has been found to help in the reduction of gingivitis, plaque, sensitivity, and to provide improved breath benefits. Polyphosphate can chelate with stannous ion in solution such that the stringent taste of the stannous can greatly be reduced. In addition, polyphosphates are effective in preventing tin deposition on teeth which results in staining. However, like polyphosphate, stannous (II) has a short life in solution, as it converts from stannous (II) to stannous (IV) easily.

Therefore, there remains a need to provide oral compositions, in particular single-phased oral compositions, in which the polyphosphate and the stannous ions are stabilized, and in which soluble fluoride ion can be efficaciously incorporated. None of the existing art provides all of the advantages and benefits of the present invention.

### SUMMARY OF THE INVENTION

The present invention relates to an oral composition comprising: (a) an effective amount of one or more linear polyphosphates having an average chain length of about 4 or more; (b) from about 0.15% to about 5% of a fluoride ion source; (c) from about 0.1% to about 15% of a stannous ion source; (d) an effective amount of a buffering agent; (e) from about 6% to about 70% of an abrasive polishing material containing less than 23% calcium; and (f) from about 40% to about 99% of one or more aqueous carriers; wherein the oral composition has a total water content of from about 1% to about 20%.

These and other features, aspects, and advantages of the present invention will become evident to those of skill in the art from a reading of the present disclosure.

### DETAILED DESCRIPTION OF THE INVENTION

All percentages and ratios used herein are by weight of the oral composition, unless otherwise specified. All measurements referred to herein are made at 25° C., unless otherwise specified.

All percentages, ratios, and levels of ingredients referred to herein are based on the actual amount of the ingredient, and do not include solvents, fillers, or other materials with which the ingredient may be combined as a commercially available product, unless otherwise specified.

All publications, patent applications, and issued patents referred to herein are incorporated herein by reference in their entireties. Citation of any reference is not an admission regarding any determination as to its availability as prior art to the claimed invention.

Herein, “comprising” means that other steps and other components which do not affect the end result can be added. This term encompasses the terms “consisting of” and “consisting essentially of.”

Herein, “effective amount” means an amount of a compound of composition sufficient to significantly induce a positive benefit, preferably an oral health benefit, but low enough to avoid serious side effects, i.e., to provide a reasonable benefit to risk ratio, within the sound judgment of a skilled artisan.

The oral composition of the present invention may be in the form of a toothpaste or dentifrice. The term “dentifrice”, as used herein, means paste, gel, or liquid formulations unless otherwise specified. The dentifrice composition may be in any desired form, such as deep striped, surface striped, multilayered, having the gel surrounding the paste, or any combination thereof. Alternatively, the oral composition may be one of the dentifrice compositions in a dual phase system comprising two dentifrice compositions contained in a physically separated compartment of a dispenser and dispensed side-by-side.

The term “dispenser”, as used herein, means any pump, tube, or container suitable for dispensing toothpaste.

The term “oral composition” as used herein means the total dentifrice that is delivered to the oral surfaces. The oral composition is a product, which in the ordinary course of usage, is not intentionally swallowed for purposes of systemic administration of particular therapeutic agents, but is rather retained in the oral cavity for a time sufficient to contact substantially all of the dental surfaces and/or oral tissues for purposes of oral activity.

The term “aqueous carrier” as used herein means any safe and effective materials for use in the compositions of the present invention. Such materials include abrasive polishing materials, peroxide sources, alkali metal bicarbonate salts, thickening materials, humectants, water, surfactants, titanium dioxide, mica, flavor systems, sweetening agents, xylitol, coloring agents, desensitizing agents, urea, and mixtures thereof.

The present compositions comprise essential components, as well as optional components. The essential and optional components of the compositions of the present invention are described in the following paragraphs.

Polyphosphate Source

The present invention includes a polyphosphate source. A polyphosphate is generally understood to consist of two or more phosphate molecules arranged primarily in a linear configuration, although some cyclic derivatives may be present. Although pyrophosphates are a polyphosphate, the polyphosphates desired are those having 4 or more phosphate molecules. The pyrophosphates are discussed separately. The inorganic polyphosphate salts desired include tetrapolyphosphate and hexametaphosphate, among others. Polyphosphates larger than tetrapolyphosphate usually occur as amorphous glassy materials. Preferred in this invention are the linear “glassy” polyphosphates having the formula:

XO(XPO₃)ₙX

wherein X is lithium, magnesium, sodium, potassium, or ammonium, preferably sodium or potassium, and n averages from about 6 to about 125. Preferred are polyphosphates manufactured by FMC Corporation which are commercially known as Sodaphos (n≈6), Hexaphos (n≈13), and Glass H (n≈21). Such materials are also available from, e.g., the Jiang Su Cheng Xing Chemical Co., Jiang Yin City, Jiang Su province, China. These polyphosphates may be used alone or in any combination thereof.

The phosphate sources are described in more detail in Kirk & Othmer, Encycl. Chemical Technology, Fourth Edition, Volume 18, Wiley-Interscience Publishers (1996). The polyphosphate source will typically comprise from about 0.5% to about 30%, preferably from about 2% to about 26%, more preferably from about 3% to about 20%, and most preferably from about 4% to about 13%, by weight of the oral composition.

Fluoride Ion Source

The dentifrice composition of the present invention incorporates a soluble fluoride ion source capable of providing free fluoride ions. Fluoride ion sources are well known for use in oral compositions as anti-caries agents. Preferred soluble fluoride ion sources herein include sodium fluoride, stannous fluoride, indium fluoride, potassium fluoride, ammonium fluoride, sodium monofluorophosphate, and mixtures thereof. Norris et al., U.S. Pat. No. 2,946,725, issued Jul. 26, 1960, and Widder et al., U.S. Pat. No. 3,678,154 issued Jul. 18, 1972, disclose such fluoride ion sources as well as others. Sodium fluoride is a highly preferred soluble fluoride ion source.

The present compositions may contain a soluble fluoride ion source capable of providing from about 50 ppm to about 3500 ppm, and preferably from about 500 ppm to about 3000 ppm of free fluoride ions. To deliver the desired amount of fluoride ions, fluoride ion sources may be present in the total oral composition at an amount of from about 0.15% to about 5%, preferably from about 0.2% to about 1%, and more preferably from about 0.2% to about 0.6%, by weight of the total composition delivered to the oral cavity.

Stannous Ion Source

The present composition includes a stannous ion source, preferably a stannous (II) ion. Stannous has been found to help in the reduction of gingivitis, plaque, sensitivity, and in improving breath benefits. The stannous provided in an oral composition will provide efficacy to a subject using the composition. Formulations providing efficacy typically include stannous levels ranging from about 3,000 ppm to about 15,000 ppm stannous ions in the composition. Below 3,000 ppm stannous the efficacy of the stannous is not sufficient. Preferably, the stannous ion is present in an amount of about 4,000 ppm to about 12,000 ppm, more preferably 5,000 ppm to about 10,000 ppm. These are ranges of stannous ions representative of a single phase oral composition. If the stannous ions were in one phase of a dual phase composition, the stannous concentration would be doubled.

Dentifrices containing stannous salts, particularly stannous fluoride and stannous chloride, are described in U.S. Pat. No. 5,004,597 to Majeti et al. Other descriptions of stannous salts are found in U.S. Pat. No. 5,578,293 issued to Prencipe et al. and in U.S. Pat. No. 5,281,410 issued to Lukacovic et al. The stannous ions herein are preferably provided from a stannous chloride, stannous chloride dihydrate, stannous fluoride, stannous sulfate, and/or other stannous salt that is added to the oral composition, with stannous chloride, stannous chloride dihydrate, stannous fluoride, stannous sulfate, and mixtures thereof being preferred. In general, stannous chloride and stannous chloride dihydrate are less costly sources of stannous as compared to stannous fluoride; because they are believed to deliver equally efficacious benefits as compared to stannous fluoride, they are preferred for use when it is desirable to manage the overall cost of the composition.

Other desirable stannous salts include stannous acetate, stannous gluconate, stannous oxalate, stannous lactate, and stannous tartrate.

In addition to the stannous ion source, other ingredients needed to stabilize the stannous may also be included, such as the ingredients described in Majeti et al. and Prencipe et al.

The combined stannous salts will be present in an amount of from about 0.1% to about 15%, by weight of the total composition. Preferably, the stannous salts are present in an amount of from about 0.5 to about 7%, more preferably from about 1% to about 5%, and most preferably from about 1.5% to about 3% by weight of the total composition.

Buffering Agent

The present composition contains a buffering agent. Buffering agents, as used herein, refer to agents that can be used to adjust the pH of the compositions to a range of about pH 6.5 to about pH 10. These agents include alkali metal hydroxides, carbonates, sesquicarbonates, borates, silicates, phosphates, imidazole, and mixtures thereof. Specific buffering agents include monosodium/monopotassium phosphate, disodium/dipotassium phosphate, trisodium/tripotassium phosphate, sodium hydroxide, potassium hydroxide, alkali metal carbonate salts, sodium carbonate, imidazole, pyrophosphate salts, citric acid, and sodium/potassium citrate. Buffering agents are used at a level of from about 0.1% to about 30%, preferably from about 1% to about 10%, and more preferably from about 0.5% to about 3%, by weight of the present composition.

Pyrophosphate Salt

Pyrophosphate salts may also be buffering agents. The pyrophosphate salts useful in the present compositions include the dialkali metal pyrophosphate salts, tetra alkali metal pyrophosphate salts, and mixtures thereof. Disodium dihydrogen pyrophosphate (Na₂H₂P₂O₇), trisodium hydrogen pyrophosphate (Na₃HP₂O₇), tetrasodium pyrophosphate (Na₄P₂O₇), and tetrapotassium pyrophosphate (K₄P₂O₇) in their unhydrated as well as hydrated forms are the preferred species. In compositions of the present invention, the pyrophosphate salt may be present in one of three ways: predominately dissolved, predominately undissolved, or a mixture of dissolved and undissolved pyrophosphate.

Compositions comprising predominately dissolved pyrophosphate refer to compositions where at least one pyrophosphate ion source is in an amount sufficient to provide at least about 1.0% free pyrophosphate ions. The amount of free pyrophosphate ions may be from about 1% to about 15%, preferably from about 1.5% to about 10%, and most preferably from about 2% to about 6%, by weight of the composition. Free pyrophosphate ions may be present in a variety of protonated states depending on a the pH of the composition.

Compositions comprising predominately undissolved pyrophosphate refer to compositions containing no more than about 20% of the total pyrophosphate salt dissolved in the composition, preferably less than about 10% of the total pyrophosphate dissolved in the composition. Tetrasodium pyrophosphate salt is the preferred pyrophosphate salt in these compositions. Tetrasodium pyrophosphate may be the anhydrous salt form or the decahydrate form, or any other species stable in solid form in the dentifrice compositions. The salt is in its solid particle form, which may be its crystalline and/or amorphous state, with the particle size of the salt preferably being small enough to be aesthetically acceptable and readily soluble during use. The amount of pyrophosphate salt useful in making these compositions is any tartar control effective amount, and is generally from about 1.5% to about 15%, preferably from about 2% to about 10%, and most preferably from about 2.5% to about 8%, by weight of the composition. Some or all of the tetrasodium pyrophosphate maybe undissolved in the product and present as tetrasodium pyrophosphate particles. Pyrophosphate ions in different protonated states (e.g., HP₂O₇⁻³) may also exist depending upon the pH of the composition and if part of the tetrasodium pyrophosphate is dissolved.

Compositions may also comprise a mixture of dissolved and undissolved pyrophosphate salts. Any of the above mentioned pyrophosphate salts may be used.

The pyrophosphate salts are described in more detail in Kirk & Othmer, Encyclopedia of Chemical Technology, Third Edition, Volume 17, Wiley-lnterscience Publishers (1982).

Optional agents to be used in place of or in combination with the pyrophosphate salt include such materials known to be effective in reducing calcium phosphate mineral deposition related to calculus formation. Agents included are synthetic anionic polymers [including polyacrylates and copolymers of maleic anhydride or acid and methyl vinyl ether (e.g., Gantrez), as described, for example, in U.S. Pat. No. 4,627,977, to Gaffar et al.; as well as, e.g., polyamino propoane sulfonic acid (AMPS)], zinc citrate trihydrate, diphosphonates (e.g., EHDP; AHP), polypeptides (such as polyaspartic and polyglutamic acids), and mixtures thereof.

Aqueous Carriers

In preparing the present compositions, it is desirable to add one or more aqueous carriers to the compositions. Such materials are well known in the art and are readily chosen by one skilled in the art based on the physical and aesthetic properties desired for the compositions being prepared. Aqueous carriers typically comprise from about 40% to about 99%, preferably from about 70% to about 98%, and more preferably from about 90% to about 95%, by weight of the oral composition.

Abrasive Polishing Materials

An abrasive polishing material is generally included in the toothpaste compositions. The abrasive polishing material contemplated for use in the compositions of the present invention can be any material which does not excessively abrade dentin. The abrasive polishing material must have a calcium content of less than 23%. Without being limited by theory, it is believed that such a calcium content is desirable to reduce interaction between the fluoride ion source and incompatible materials present in the abrasive polishing material, e.g., calcium. Typical abrasive polishing materials include silicas including gels and precipitates; aluminas; phosphates including orthophosphates, polymetaphosphates, and pyrophosphates; and mixtures thereof. Specific examples include dicalcium orthophosphate dihydrate, calcium pyrophosphate, tricalcium phosphate, calcium polymetaphosphate, insoluble sodium polymetaphosphate, hydrated alumina, beta calcium pyrophosphate, calcium carbonate, and resinous abrasive materials such as particulate condensation products of urea and formaldehyde, and others such as disclosed by Cooley et al in U.S. Pat. No. 3,070,510, issued Dec. 25, 1962. Mixtures of abrasives may also be used. Only the abrasive polishing materials containing less than 23% calcium may be used in a single phase system herein.

Silica dental abrasives of various types are preferred because of their unique benefits of exceptional dental cleaning and polishing performance without unduly abrading tooth enamel or dentine. The silica abrasive polishing materials herein, as well as other abrasives, generally have an average particle size ranging between about 0.1 to about 30 microns, and preferably from about 5 to about 15 microns. The abrasive can be precipitated silica or silica gels such as the silica xerogels described in Pader et al., U.S. Pat. No. 3,538,230, issued Mar. 2, 1970, and DiGiulio, U.S. Pat. No. 3,862,307, issued Jan. 21, 1975. Preferred are the silica xerogels marketed under the trade name “Syloid” by the W. R. Grace & Company, Davison Chemical Division. Also preferred are the precipitated silica materials such as those marketed by the J. M. Huber Corporation under the trade name, “Zeodent”, particularly the silica carrying the designation “Zeodent 119”. The types of silica dental abrasives useful in the toothpastes of the present invention are described in more detail in Wason, U.S. Pat. No. 4,340,583, issued Jul. 29, 1982. Silica abrasives are also described in U.S. patent application Ser. Nos. 08/434,147 and 08/434,149, both filed May 2, 1995. The abrasive in the toothpaste compositions described herein is generally present at a level of from about 6% to about 70% by weight of the composition. Preferably, toothpastes contain from about 10% to about 50% of abrasive, by weight of the oral composition.

Humectant

Another preferred aqueous carrier desired herein is a humectant. The humectant serves to keep toothpaste compositions from hardening upon exposure to air and certain humectants can also impart desirable sweetness of flavor to toothpaste compositions. Suitable humectants for use in the invention include glycerin, sorbitol, polyethylene glycol, propylene glycol, and other edible polyhydric alcohols. The humectant generally comprises from about 0% to 70%, and preferably from about 15% to 55%, by weight of the composition.

Herein, glycerin, polyethylene glycol, propylene glycol, and mixtures thereof are the preferred humectants. It is known that the polyphosophate and the stannous ion sources herein decompose in the presence of water. It has been discovered that if the polyphosophate and the stannous ion sources herein are dispersed in the humectant during manufacture, the stability of these components can be maintained in the product form. Since the overall amount of water contained in the composition is low, i.e., from about 1% to about 20%, preferably from about 2% to about 14%, and more preferably from about 3% to about 10%, dispersing both the polyphosphate and the stannous source in the matrix ensures the stability of each of these two components.

As used herein, “stable” refers to the amount of total soluble fluoride content, the amount of total soluble stannous content, and the amount of released orthophosphate from the polyphosphate hydrolysis of the composition. To determine if the composition is stable, the total soluble fluoride and total soluble stannous present in the composition may be measured by methods known to those of skill in the art. If there is a significant loss of soluble fluoride and/or soluble stannous, the composition is not stable.

The amount of orthophosphate can be measured, e.g., using 31P-NMR method. If the amount of orthophosphate is not significantly increased over a certain period at a certain temperature, the polyphosphate is stable. For example, it is believed that the level of orthophosphate released as a result of polyphosphate hydrolysis in the compositions herein is not greater than 2% after 3 months at room temperature.

Additionally, the appearance of a white precipitate may also be a measure of stability. Appearance of a white precipitate indicates that the composition is not stable. Without being limited by theory, it is believed that the compositions herein will exhibit stability during ordinary storage and shelf life conditions of time and temperature.

In this manner, an efficacious, stable, and single-phased dentifrice composition can be provided. Because there is no need to keep those components that decompose in water separated from those that are stable in water until just prior to use, such a composition need not be dispensed from a dual compartment dispenser. Thus, the composition can efficaciously deliver oral care actives without increasing the cost associated with packaging.

This being said however, it should be understood that the present invention is not limited to a single-phased composition. The present invention includes dual-phased compositions in which two oral formulations are contained in physically separate compartments of a dentifrice dispenser. In general, the polyphosphate should be separated from the fluoride and stannous ion sources. By way of example, one of the two oral formulations comprises the polyphosphate, the stannous ion source, an effective amount of a buffering agent, and one or more aqueous carriers, the total water content being from about 1% to about 20%, preferably from about 2% to about 14%, and more preferably from about 3% to about 10%; and the other oral formulation generally comprises the fluoride ion source, an effective amount of a buffering agent, and one or more aqueous carriers; the total water content being from about 1% to about 20%.

Peroxide Source

The present compositions may include a peroxide source. The peroxide source is preferably selected from the group consisting of hydrogen peroxide, calcium peroxide, urea peroxide, and mixtures thereof. The following amounts represent the amount of peroxide raw material, although the peroxide source may contain ingredients other than the peroxide raw material. The present composition may contain from about 0.01 % to about 10%, preferably from about 0.1% to about 5%, more preferably from about 0.2% to about 3%, and most preferably from about 0.3% to about 0.8% of a peroxide source, by weight of the dentifrice composition.

Alkali Metal Bicarbonate Salt

The present invention may also include an alkali metal bicarbonate salt. Alkali metal bicarbonate salts are soluble in water and unless stabilized, tend to release carbon dioxide in an aqueous system. Sodium bicarbonate, also known as baking soda, is the preferred alkali metal bicarbonate salt. The alkali metal bicarbonate salt also functions as a buffering agent. The present composition may contain from about 0.5% to about 50%, preferably from about 0.5% to about 30%, more preferably from about 2% to about 20%, and most preferably from about 5% to about 18% of an alkali metal bicarbonate salt, by weight of the oral composition.

Additional Aqueous Carriers

The present invention compositions in the form of toothpastes, typically contain some thickening material or binders to provide a desirable consistency. Preferred thickening agents are carboxyvinyl polymers, carrageenan, hydroxyethyl cellulose, and water soluble salts of cellulose ethers such as sodium carboxymethylcellulose and sodium hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture. Thickening agents can be used in an amount from about 0.1% to about 15%, by weight of the dentifrice composition.

Water employed in the preparation of commercially suitable oral compositions should preferably be of low ion content and free of organic impurities. The dentifrice composition will contain a water of from about 1% to about 20%, preferably from about 2% to about 14%, and more preferably from about 3% to about 10%, by weight of the composition. The amounts of water include the free water which is added plus that which is introduced with other materials, such as with sorbitol, silica, surfactant solutions, and/or color solutions. Preferably, no free water is added to the compositions herein. Without being limited by theory, it is believed that the minimization of water in the compositions herein reduces the negative interaction between polyphosphate and fluoride, slows down polyphosphate hydrolysis, as well as stabilizing stannous ion.

The present compositions may also comprise surfactants, also commonly referred to as sudsing agents. Suitable surfactants are those which are reasonably stable and foam throughout a wide pH range. The surfactant may be anionic, nonionic, amphoteric, zwitterionic, cationic, or mixtures thereof. Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed. Many suitable anionic surfactants are disclosed by Agricola et al., U.S. Pat. No. 3,959,458, issued May 25, 1976. Nonionic surfactants which can be used in the compositions of the present invention can be broadly defined as compounds produced by the condensation of alkylene oxide groups (hydrophilic in nature) with an organic hydrophobic compound which may be aliphatic or alkyl—aromatic in nature. Examples of suitable nonionic surfactants include poloxamers (sold under trade name Pluronic), polyoxyethylene, polyoxyethylene sorbitan esters (sold under trade name Tweens), fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, and mixtures of such materials. The amphoteric surfactants useful in the present invention can be broadly described as derivatives of aliphatic secondary and tertiary amines in which the aliphatic radical can be a straight chain or branched and wherein one of the aliphatic substituents contains from about 8 to about 18 carbon atoms and one contains an anionic water-solubilizing group, e.g., carboxylate, sulfonate, sulfate, phosphate, or phosphonate. Other suitable amphoteric surfactants are betaines, specifically cocamidopropyl betaine. Mixtures of amphoteric surfactants can also be employed. Many of these suitable nonionic and amphoteric surfactants are disclosed by Gieske et al., U.S. Pat. No. 4,051,234, issued Sep. 27, 1977. The present composition typically comprises one or more surfactants each at a level of from about 0.25% to about 12%, preferably from about 0.5% to about 8%, and most preferably from about 1% to about 6%, by weight of the composition.

Titanium dioxide may also be added to the present composition. Titanium dioxide is a white powder which adds opacity to the compositions. Titanium dioxide generally comprises from about 0.25% to about 5%, by weight of the composition. Similarly, mica may added to the present compositions in order to provide opacity and to further provide a shimmery or glittery appearance. Mica generally comprises from about 0.1% to about 5%, by weight of the composition.

Coloring agents may also be added to the present composition. The coloring agent may be in the form of an aqueous solution, preferably 1% coloring agent in a solution of water, or in the form of pigments. Color solutions generally comprise from about 0.01% to about 5%, by weight of the composition.

A flavor system can also be added to the compositions. Suitable flavoring components include oil of wintergreen, oil of peppermint, oil of spearmint, eucalyptus oil, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) and mixtures thereof. A flavor system is generally used in the compositions at levels of from about 0.001% to about 5%, by weight of the composition.

The present invention may also include xylitol. Xylitol is a sugar alcohol that is used as a sweetener and humectant. Xylitol may provide a therapeutic effect, such as an antibacterial or anticaries effect. The present compositions typically comprise xylitol at a level from about 0.01% to about 25%, preferably from about 3% to about 15%, more preferably from about 5% to about 12%, and most preferably from about 9% to about 11%, by weight of the total composition. Alternatively, if xylitol is used as a sweetener, it may be present at a lower level, such as from about 0.005% to about 5%, by weight of the dentifrice composition.

Sweetening agents can be added to the compositions. These include saccharin, dextrose, sucrose, lactose, maltose, levulose, aspartame, sodium cyclamate, D-tryptophan, dihydrochalcones, acesulfame, and mixtures thereof. Various coloring agents may also be incorporated in the present invention. Sweetening agents and coloring agents are generally used in toothpastes at levels of from about 0.005% to about 5%, by weight of the composition.

The present invention may also include other agents, such as antimicrobial agents. Included among such agents are water insoluble non-cationic antimicrobial agents such as halogenated diphenyl ethers, phenolic compounds including phenol and its homologs, mono and poly-alkyl and aromatic halophenols, resorcinol and its derivatives, bisphenolic compounds and halogenated salicylanilides, benzoic esters, and halogenated carbanilides. The water soluble antimicrobials include quaternary ammonium salts and bis-biquanide salts, among others. Triclosan monophosphate is also a suitable water soluble antimicrobial agent. The quaternary ammonium agents include those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms, typically methyl or ethyl groups. Dodecyl trimethyl ammonium bromide, tetradecylpyridinium chloride, domiphen bromide, N-tetradecyl-4-ethyl pyridinium chloride, dodecyl dimethyl (2-phenoxyethyl) ammonium bromide, benzyl dimethylstearyl ammonium chloride, cetyl pyridinium chloride, quaternized 5-amino-1,3-bis(2-ethyl-hexyl)-5-methyl hexa hydropyrimidine, benzalkonium chloride, benzethonium chloride and methyl benzethonium chloride are exemplary of typical quaternary ammonium antibacterial agents. Other compounds are bis[4-(R-amino)-1-pyridinium] alkanes as disclosed in U.S. Pat. No. 4,206,215, issued Jun. 3, 1980, to Bailey. Stannous salts such as stannous pyrophosphate and stannous gluconate and other antimicrobials such as copper bisglycinate, copper glysinate, zinc citrate, and zinc lactate may also be included. Also useful are enzymes, including endoglycosidase, papain, dextranase, mutanase, and mixtures thereof. Such agents are disclosed in U.S. Pat. No. 2,946,725, Jul. 26, 1960, to Norris et al. and in U.S. Pat. No. 4,051,234, Sep. 27, 1977 to Gieske et al. Specific antimicrobial agents include chlorhexidine, triclosan, triclosan monophosphate, and flavor oils such as thymol. Triclosan and other agents of this type are disclosed in Parran, Jr. et al., U.S. Pat. No. 5,015,466, issued May 14, 1991, and U.S. Pat. No. 4,894,220, Jan. 16, 1990 to Nabi et al. These agents may be present at levels of from about 0.01% to about 1.5%, by weight of the composition.

Urea may also be present in the compositions herein. Without being limited by theory, it is believed that urea acts as a penetration agent to help the actives herein better diffuse into the tooth enamel and/or the gum tissue. Urea may be present at levels of from about 0.2% to about 5% by weight of the composition.

A effective amount of a desensitizing agent may also be incorporated in the compositions herein. The desensitizing agents include those selected from alkaline metal salts with a chloride, nitrate, sulfate, or acetate of a group ll metal or aluminum or polymerizable monomer to occlude the tubules, alkaline metal or ammonium oxylate, citric acid and sodium citrate. Preferred salts are potassium nitrate, potassium citrate, and mixtures thereof. Such desensitizing agents are disclosed in, e.g., U.S. Pat. No. 5,718,885, issued Feb. 17, 1998 to Gingold et al.

Method of Treatment

The present invention compositions additionally relate to a method for reducing the incidence of calculus on dental enamel of a human or animal, e.g., household pets or other domestic animals, or animals kept in captivity. The method of treatment herein comprises contacting the dental enamel surfaces in the mouth with the oral compositions according to the present invention.

Examples & Method of Manufacturing

The following examples further describe and demonstrate embodiments within the scope of the present invention. These examples are given solely for the purpose of illustration and are not to be construed as limitations of the present invention as many variations thereof are possible without departing from the spirit and scope.

### EXAMPLES 1-6

The following examples are prepared according to the method below.

| Component | Ex. 1 | Ex. 2 | Ex. 3 | Ex. 4 | Ex. 5 | Ex. 6 |
|---|---|---|---|---|---|---|
| Glass H | 13 | 13 | 13 | 13 | 13 | 13 |
| Polyphosphate | | | | | | |
| Sodium | 0.243 | 0.243 | 0.243 | 0.243 | 0.243 | 0.243 |
| Fluoride | | | | | | |
| Stannous | 2.1 | 2.1 | 2.1 | 2.1 | 1.43 | 1.43 |
| Chloride | | | | | | |
| Dihydrate | | | | | | |
| Sodium Citrate | 4 | — | — | 4 | — | 6 |
| Sodium | 2 | — | — | 3 | — | — |
| Dicarbonate | | | | | | |
| Sodium | — | 0.8 | — | — | — | 0.15 |
| Hydroxide | | | | | | |
| Tetrasodium | — | — | 4 | — | 4 | — |
| Pyrophosphate | | | | | | |
| Silica (Zeodent | 21 | 21 | 21 | 21 | 21 | 23.322 |
| 119) | | | | | | |
| Urea | — | — | — | — | 1 | — |
| Glycerin | 27.451 | 29.451 | 29.251 | 29.451 | 29.451 | 29.451 |
| PEG-300 | 6 | 7 | 9 | 7 | 8.47 | 7 |
| Propylene | 7 | 9 | 9 | 9 | 9 | 7 |
| Glycol | | | | | | |
| Xylitol | 5 | 5 | — | — | — | — |
| Poloxamer | 3 | 3 | 3 | 3 | 3 | 3 |
| Sodium Alkyl | 2.09 | 2.09 | 2.09 | 2.09 | 2.09 | 2.09 |
| Sulfate | | | | | | |
| 27.9% Solution | | | | | | |
| Mica | 0.2 | 0.2 | 0.2 | 0.2 | 0.2 | 0.2 |
| Acesulfame K | — | 0.2 | 0.2 | — | 0.2 | 0.2 |
| Flavor | 1.1 | 1.1 | 1.1 | 1.1 | 1.1 | 1.1 |
| Saccharin | 0.4 | 0.4 | 0.4 | 0.4 | 0.4 | 0.4 |
| Blue Dye No. 1 | 0.006 | 0.006 | 0.006 | 0.006 | 0.006 | 0.004 |
| D.I. Water | 5.41 | 5.41 | 5.41 | 5.41 | 5.41 | 5.41 |
| TOTAL | 100 | 100 | 100 | 100 | 100 | 100 |

### EXAMPLES 7-11

The following examples are prepared according to the method below.

| Component | Ex. 7 | Ex. 8 | Ex. 9 | Ex. 10 | Ex. 11 |
|---|---|---|---|---|---|
| Glass H Polyphosphate | 13 | 13 | — | 13 | 13 |
| Sodaphos Polyphosphate | — | — | 13 | — | — |
| Sodium Fluoride | 0.243 | 0.243 | 0.243 | 0.243 | 0.243 |
| Sodium | — | — | — | — | — |
| Monofluorophosphate | | | | | |
| Stannous Chloride | 1.43 | 1.43 | 1.43 | 1.43 | 1.43 |
| Carboxymethycellulose | 0.3 | 0.6 | 0.6 | 0.6 | 0.6 |
| Water | 5.41 | 5.41 | 5.41 | 5.41 | 5.41 |
| Flavor | 1.1 | 1.1 | 1.1 | 1.1 | 1.1 |
| Glycerin | 34.327 | 27.527 | 22.027 | 27.57 | 30.927 |
| Poloxamer 407 | 6 | 3 | 5 | 5 | 3 |
| Propylene Glycol | 10 | 5 | 5 | 3 | 3 |
| Sodium Alkyl Sulfate | 2.09 | 2.09 | 2.09 | 4 | 2.09 |
| 27.9% Solution | | | | | |
| Silica | 10 | 22 | 20 | 17.547 | 27 |
| Sodium Bicarbonate | — | 15 | 15 | 15 | 5 |
| Sodium Carbonate | 2 | 2 | 2 | 2 | 2 |
| Sodium Saccharin | 0.4 | 0.5 | 0.4 | 0.4 | 0.5 |
| Titanium Dioxide | 0.5 | 0.5 | 0.5 | 0.5 | 0.5 |
| Xanthan Gum | 0.2 | 0.2 | 0.2 | 0.2 | 0.2 |
| Polyethylene Glycol | 12 | — | 3 | 3 | 3 |
| Calcium Peroxide | 1 | 0.4 | 3 | — | 1 |
| TOTAL | 100 | 100 | 100 | 100 | 100 |

### EXAMPLES 12-16

The following examples are prepared according to the method below.

| Component | Ex. 12 | Ex. 13 | Ex. 14 | Ex. 15 | Ex. 16 |
|---|---|---|---|---|---|
| Glass H Polyphosphate | 13 | 13 | — | 13 | 13 |
| Sodaphos Polyphosphate | — | — | 13 | — | — |
| Sodium Fluoride | — | — | — | — | — |
| Sodium | 0.76 | 0.76 | 0.76 | 0.76 | 0.76 |
| Monofluorophosphate | | | | | |
| Stannous Chloride | 1.43 | 1.43 | 1.43 | 1.43 | 1.43 |
| Carboxymethycellulose | 0.6 | 0.6 | 0.6 | 0.6 | 0.6 |
| Water | 5.41 | 5.41 | 5.41 | 5.41 | 5.41 |
| Flavor | 1.1 | 1.1 | 1 | 1 | 1 |
| Glycerin | 25.44 | 25.44 | 25.44 | 40.51 | 25.01 |
| Poloxamer 407 | 5 | 5 | 5 | 5 | 5 |
| Propylene Glycol | 3 | 3 | 3 | 3 | 3 |
| Sodium Alkyl Sulfate | 2.09 | 2.09 | 2.09 | 2.09 | 4 |
| 27.9% Solution | | | | | |
| Silica | 20.07 | 20.07 | 20.17 | 20 | 19.69 |
| Sodium Bicarbonate | 15 | 15 | 15 | — | 15 |
| Sodium Carbonate | 2 | 2 | 2 | 2 | 2 |
| Sodium Saccharin | 0.4 | 0.4 | 0.4 | 0.5 | 0.4 |
| Titanium Dioxide | 0.5 | 0.5 | 0.5 | 0.5 | 0.5 |
| Xanthan Gum | 0.2 | 0.2 | 0.2 | 0.2 | 0.2 |
| Polyethylene Glycol | 3 | 3 | 3 | 3 | 3 |
| Calcium Peroxide | 1 | 1 | 1 | 1 | — |
| TOTAL | 100 | 100 | 100 | 100 | 100 |

The dentifrice compositions are prepared as follows. Add the sodium alkyl sulfate solution, fluoride salts, sweeteners, and coloring agents to the main mix tank. Agitate at the rate of approximately 44±4 rpm, and heat to about 40±5° C. Maintain for about 20 minutes. Add about ⅓ of the glycerin to the main mix tank, then add the poloxamer and homogenize for about 20 minutes. Add the remaining glycerin to the main mix tank. Add the propylene glycol and PEG to a mixing vessel. In the main mix tank, start agitator to pull a vortex for good mixing and homogenize for 5 minutes. Then decrease the temperature to approximately 35° C. Add the silica to the main mix tank. After the addition of the silica, completely open the vacuum to about 0.1 to 0.2 bar for 10 minutes. Add the flavor into the mixing vessel. Premix the polyphosphate, the stannous salts, calcium peroxide (if used) and sodium bicarbonate (if used), and the buffering agent, then add into the main mix tank. Stir and homogenize for 20 minutes, then vacuum for 10 minutes at the end of the homogenization.

It is understood that the examples and embodiments described herein are for illustrative purposes only and that various modifications or changes in light thereof will be suggested to one skilled in the art without departing from the scope of the present invention.

What is claimed is:

1. An oral composition comprising:
a. an effective amount of one or more linear polyphosphates having an average chain length of about 4 or more;
b. from about 0.15% to about 5% of a fluoride ion source;
c. from about 0.1% to about 15% of a stannous ion source;
d. an effective amount of a buffering agent;
e. from about 6% to about 70% of an abrasive polishing material containing less than 23% calcium; and
f. from about 50% to about 99% of one or more aqueous carriers;
wherein the oral composition has a total water content of from about 1% to about 20%.

2. The oral composition according to claim 1 wherein the one or more polyphosphates have an average chain length of 6 or more.

3. The oral composition according to claim 2 wherein each of the one or more polyphosphates is present in an amount of from about 0.5% to about 30%.

4. The oral composition according to claim 3 wherein each of the one or more polyphosphates is selected from the group consisting of linear “glassy” polyphosphates having the formula
XO(XPO₃)ₙX
wherein X is lithium, magnesium, sodium, potassium, or ammonium and n averages from about 6 to about 21.

5. The oral composition according to claim 1 wherein the fluoride ion source is sodium fluoride.

6. The oral composition according to claim 1 wherein the stannous ion source is selected from the group consisting of stannous chloride, stannous chloride dihydrate, stannous fluoride, stannous sulfate, and mixtures thereof.

7. The composition according to claim 1 wherein the abrasive polishing material is selected from the group consisting of silicas, aluminas, phosphates, orthophosphates, polymetaphosphates, and mixtures thereof.

8. The oral composition according to claim 1 wherein the aqueous carriers are materials selected from the group consisting of humectants, peroxide sources, alkali metal bicarbonate salts, surfactants, thickening materials, water, titanium dioxide, mica, flavor systems, sweetening agents, xylitol, coloring agents, urea, desensitising agents, and mixtures thereof.

9. The oral composition according to claim 1 wherein the humectant is selected from the group consisting of glycerin, propylene glycol, polyethylene glycol, and mixtures thereof.

10. The oral composition according to claim 1 wherein the composition is a single-phased dentifrice composition.

11. The oral composition according to claim 10 wherein the oral composition has a total water content of from about 2% to about 14%.

12. The oral composition according to claim 11 wherein the oral composition has a total water content of from about 3% to about 10%.

13. A method for reducing the incidence of calculus on dental enamel comprising contacting the enamel surfaces in the mouth with the oral composition according to claim 1.
